(19) United States
(12) Patent Application Publication
(10) Pub. No.: US 2024/0099696 A1
(43) Pub. Date: Mar. 28, 2024

(54) ULTRASOUND ENDOSCOPY

(71) Applicant: AMBU A/S, Ballerup (DK)

(72) Inventors: Søren Elmin DIEDERICHSEN, Frederiksberg (DK); Finn SONNENBORG, Frederikssund (DK); Morten SØRENSEN, Ballerup (DK)

(73) Assignee: AMBU A/S, Ballerup (DK)

(21) Appl. No.: 18/372,993

(22) Filed: Sep. 26, 2023

(30) Foreign Application Priority Data
Sep. 27, 2022 (EP) EP22198066.7
Sep. 27, 2022 (EP) EP22198073.3
Sep. 27, 2022 (EP) EP22198097.2

(51) Int. Cl. A61B 8/00; A61B 8/12; B06B 1/06

(52) U.S. Cl. CPC A61B 8/4488; A61B 8/12; A61B 8/4416; B06B 1/0622; B06B 1/067; B06B 2201/76

(57) ABSTRACT

A curvilinear ultrasound transducer and a method of manufacturing the same. The method includes providing a piezoelectric block having an upper side and a lower side opposite to the upper side; cutting the piezoelectric block at a plurality of positions forming a plurality of ultrasound transducer elements, the plurality of ultrasound transducer elements separated by a plurality of gaps; allowing the plurality of gaps to be filled with a first gas resulting in a plurality of gas-filled gaps; arranging an acoustic matching layer facing the upper side of the piezoelectric block; arranging the piezoelectric block and the first acoustic matching layer between a curvilinear upper surface of a support structure and a pressure element; and pressing the piezoelectric block and the first acoustic matching layer with the support structure and the pressure element to curvilinearly shape the piezoelectric block and first acoustic matching layer.

### CROSS-REFERENCE TO RELATED APPLICATIONS

This application claims priority from and the benefit of European Patent Application No. EP22198066.7, filed Sep. 27, 2022, European Patent Application No. EP22198073.3, filed Sep. 27, 2022 and European Patent Application No. EP22198097.2, filed Sep. 27, 2022; the disclosures of the aforementioned applications are incorporated by reference herein in their entirety.

### TECHNICAL FIELD

The present disclosure relates to an ultrasound transducer for an endoscope. More specifically, the disclosure relates to a method of manufacturing a curvilinear ultrasound transducer, a printed flexible electrical circuit for an ultrasound transducer, a curvilinear ultrasound transducer, and an endoscope with an ultrasound transducer.

### BACKGROUND

Examination of human cavities, such as human airways, with an endoscope may be carried out to determine whether a patient has disease, a tumor, an infection, or the like, and in some cases samples may be taken/removed from the human cavity. For instance, bronchoscopies or colonoscopies may be carried out to examine whether a patient has a lung or colon disease, respectively, a tumour, or the like. The endoscope typically comprises an image sensor, such as a camera, at a distal end of the endoscope to be inserted into the patient and connected to a display so as to provide the medical personnel with a view of the part of the airways, in which the distal end of the endoscope is positioned.

The image sensor, or image capturing device, has limited penetration depth of visible light. An endoscope with an ultrasound transducer, or sensor, can sense more deeply. Ultrasound probes are, however, more complex to manufacture an endoscope comprising an ultrasound sensor and significantly more expensive than regular endoscopes. For example, a curvilinear ultrasound transducer may comprise more than 32 ultrasound transducer elements, each of which is connected by electrical conductors to an ultrasound controller to send and receive electrical signals. Connecting the conductors to the elements is a difficult and time-consuming task. This is one of the reasons why it is complex and expensive to manufacture a curvilinear ultrasound transducer.

Furthermore, endoscopes are difficult to clean and there exist the risk of cross-contamination. Single-use endoscopes eliminate the cross-contamination risk. But the provision of an ultrasound probe at the distal end of the endoscope makes it economically difficult to provide single-use ultrasound endoscopes.

### BRIEF DESCRIPTION OF THE DISCLOSURE

The tasks and objectives of the present disclosure are to eliminate or at least to reduce the disadvantages of the related art. In particular, problems addressed by the present disclosure include how to reduce the cost of a single-use ultrasound endoscope and how to reduce the cost sufficiently to enable high volume production and deployment so that single-use endoscopes can be used regardless of the economic circumstances of the patients.

According to a first aspect, the present disclosure relates to a method of manufacturing a curvilinear ultrasound transducer for use with an endoscope. In one embodiment, the method comprises cutting a piezoelectric block at a plurality of positions on an upper side of the piezoelectric block to form a plurality of ultrasound transducer elements separated by a plurality of gaps; allowing the plurality of gaps to be filed with a first gas resulting in a plurality of gas-filled gaps; arranging a first acoustic matching layer over the upper side of the piezoelectric block; and curving the piezoelectric block and the first acoustic matching layer with a curvilinear surface of a support structure to form the curvilinear ultrasound transducer.

Using a gas to fill the plurality of gaps between the transducer elements instead of a traditional kerf filler material permits shaping of the transducer block and the acoustic matching layer. This may simplify the process of manufacturing a curvilinear ultrasound transducer. Specifically, the number of process steps may be reduced. Furthermore, it may become simpler to produce the first acoustic matching layer and, potentially, additional layers. It may also become simpler to arrange the first acoustic matching layer and additional layers on the piezoelectric block.

The piezoelectric block may be made of any kind of piezoelectric material such as lead zirconate titanate (PZT), Quartz, Barium titanate, Poly(vinylidene fluoride) (PVDF), or a piezoelectric composite material. The upper surface of the piezoelectric block may extend in a first reference plane and the lower surface of the piezoelectric block may extend in a second reference plane, before the piezo electric block has been curvilinear shaped. The first reference plane and the second reference plane may be parallel.

The first acoustic matching layer is configured to provide an acoustic impedance gradient between the acoustic impedance of the tissue and the acoustic impedance of the ultrasound transducer elements. The first acoustic matching layer may be made of epoxy, polyurethane, or polystyrene. Additional acoustic matching layers may be provided e.g. a second acoustic matching layer and a third acoustic matching layer. Potential additional acoustic matching layers are preferably also arranged above the piezo electric block before the piezo electric block has been curvilinear shaped.

The pressure in the gas-filed gaps at room temperature (20 degrees Celsius) may be lower than 1 Atmosphere (101326 Pa) e.g. the pressure may be below 0.8 Atmosphere, 0.5 Atmosphere, or 0.1 Atmosphere at room temperature (20 degrees Celsius). This may limit the transfer of acoustic energy between the transducer elements.

The positions where the piezoelectric block is may be predetermined.

The pressure element may comprise a concave surface. The concave surface may be formed by a single curved surface or a plurality of piecewise flat surfaces together forming the concave surface. Alternatively, the pressure element may be a roller pressure element starting from one end and applying pressure while rolling over to the other end.

The gas filled gaps may be sealed to prevent the pressure from increasing. The first acoustic matching layers, the support structure or other elements of the ultrasound transducer may assist in sealing the gas filed gaps. Additionally/alternatively, the piezoelectric block may be encapsulated e.g. in an epoxy resin or the like.

The support structure and the pressure element may be moved together by having the support structure being kept stationary e.g. support by an external support, and moving the pressure element toward the support structure. Alternatively, the pressure element may be kept stationary and the support structure may be moved towards the pressure element. Alternatively, the support structure may be moved towards the pressure element and the pressure element may be moved towards the support structure. The pressure element may apply a pressure between 0.5 bar and 5 bar or between 0.8 bar and 2.5 bar. As an example, the pressure may be 1 bar.

The piezoelectric block may be heated before, while and/or after the support structure and the pressure element are moved together. The piezoelectric block may be heated to a temperature between 40 degrees and 120 degrees or 60 and 100 degrees Celsius before the support structure and the pressure element are moved together. The piezoelectric block may be heated to a temperature between 25 degrees and 90 degrees or 30 and 50 degrees Celsius after the support structure and the pressure element have been moved together. As an example, the piezoelectric block may be heated to a temperature of 80 degrees for one hour, then a uniaxial pressure of 1 bar is applied using the pressure element, and finally the piezoelectric block is cured for 12 hours at 40 degrees Celsius.

The piezoelectric block is preferably permanently attached to the support structure. An adhesive may be applied between the piezoelectric block and the curvilinear upper surface of the support structure. As an example, the adhesive may be a two-component epoxy glue. The piezo electric block is preferably permanently attached to the support structure when the support structure and the pressure element are moved together.

The support structure is preferably made of a material configured to absorb ultrasound energy. The material may be a composite material. As an example, the material may be a epoxy mixed with tungsten powder.

The first gas may consist of a single type of gas or a combination of different types of gases. The first gas preferably stays in the gaseous phase at least within a normal temperature range such as from −10 degrees Celsius to +90 degrees Celsius.

In some embodiments, the first gas is atmospheric air.

In some embodiments, the first acoustic matching layer is arranged above the plurality of ultrasound transducer elements after the piezoelectric block has been cut forming the plurality of ultrasound transducer elements. Consequently, the first acoustic matching layer may structurally stabilize the ultrasound transducer elements. This may also make the step of cutting the ultrasound transducer elements simpler.

In some embodiments, the method further comprises providing a common ground electrode; and arranging the common ground electrode above the plurality of ultrasound transducer elements and subsequently arranging the first acoustic matching layer above the common ground electrode. Consequently, the ultrasound transducer may be shaped curvilinear with the common ground electrode arranged above the plurality of ultrasound transducer elements.

A layer of adhesive may be provided between the ultrasound transducer elements and the common ground electrode and/or layer of adhesive may be provided between common ground electrode and the first acoustic matching layer. The common ground electrode may be a copper ground electrode.

In some embodiments, the first acoustic matching layer is arranged above the piezoelectric block before the piezoelectric block has been cut. Consequently, transfer of acoustic energy between transducer elements may be limited.

In some embodiments, the method further comprises providing a common ground electrode, and arranging the common ground electrode above the piezoelectric block and subsequently arranging the first acoustic matching layer above the common ground electrode.

The common ground electrode may be arranged on top side of the upper side of the piezoelectric block. An adhesive may be provided between the common ground electrode and the upper side of the piezo electric block. The common ground electrode may have a width being wider than the width of the upper side of the piezoelectric block, whereby a subsequent cutting of the piezo electric block may be done while keeping all parts of the common ground electrode electrically connected.

In some variations of the embodiments according to the first aspect, the method further comprises providing a flexible electrical circuit for electrically receiving and providing electrical signals to the plurality of ultrasound transducer elements, wherein the flexible electrical circuit comprising a plurality of first electrical contacts, each first electrical contact being connectable to an ultrasound transducer element for providing and receiving electrical signals to the ultrasound transducer element; and attaching the flexible electrical circuit to the lower side of the piezoelectric block before moving the support structure and pressure element together. Using a flexible electrical circuit facilitates providing the piezo-electric block with electrical contacts for the ultrasound transducer elements before the piezo-electric block is curvilinearly shaped.

The flexible electrical circuit may be a flexible printed circuit. An adhesive may be provided between the lower side of the piezoelectric block and the flexible electrical circuit.

In some embodiments, the flexible electrical circuit is attached to the lower side of the piezoelectric block before the piezoelectric block has been cut, and wherein cutting of the piezoelectric block electrically insulates the plurality of first electrical contacts.

Before cutting, the plurality of first electrical contacts are electrically connected via the piezoelectric block. However, after cutting each first electrical contact is preferably connected to only a single transducer element.

Only the piezoelectric block may be cut, i.e. the flexible electrical circuit may be left substantially intact. This may allow the flexible electrical circuit to assist in stabilizing the plurality of ultrasound transducer elements before they are supported by the support structure.

In some embodiments, the method further comprises obtaining an acoustic lens, the acoustic lens being formed as a single element, wherein the acoustic lens has a lower surface having a concave curvature; and arranging the acoustic lens above the first acoustic matching layer. Consequently, the acoustic lens may mechanically stabilize the curvilinear ultrasound transducer with the gas-filed gaps.

According to a second aspect, the present disclosure relates to a flexible electrical circuit for receiving and providing electrical signals to the plurality of ultrasound transducer elements, the printed flexible electrical circuit comprising: a flexible base section comprising a plurality of first electrical contacts, each first electrical contact being connectable to an ultrasound transducer element for providing and receiving electrical signals to the ultrasound transducer element and extending along a central axis being parallel to a first axis; a plurality of first flexible electrical conductors, each first flexible electrical conductor being electrically connected to a first electrical contact; a contact section comprising a plurality of second electrical contacts each second electrical contact being electrically connected to a first electrical conductor and further electrical connected or connectable to a second electrical conductor or a transducer port of an ultrasound processing apparatus, wherein the plurality of first flexible electrical conductors is bendable around a support structure at a first curve section and at a second curve section, the first curve section being separate from the second curve section.

Consequently, by providing an electrical circuit with a flexible base section having a plurality of electrical contacts, a plurality of ultrasound transducer elements may be provided with an electrical connection in a single process step. This makes it simple to manufacture and thus reduces manufacturing costs. Additionally, by providing the electrical circuit with a plurality of flexible electrical conductors bendable around a support structure at a first curve section and second curve section, a single electrical circuit may be used to transport the electrical signal to and from the plurality of electrical contacts.

The flexible printed circuit may be made using Photolithography. An adhesive may be provided between the lower side of the ultrasound transducer array and the flexible base section. Correspondingly, an adhesive may be provided between the flexible base section and the upper surface of the support structure. After attachment to the plurality of ultrasound transduce elements the flexibility of the flexible base section is limited by the flexibility of the ultrasound transducer array.

In some embodiments according to a second aspect, the plurality of first flexible electrical conductors for a first part of their length extends inside the insertion tube of an endoscope according to the fourth aspect, for a second part of their length extends inside the handle of the endoscope, and for a third part of their length extend inside the first flexible cable of the endoscope.

In some embodiments according to a second aspect, the first flexible cable comprises an ultrasound connector insertable into a transducer port of an ultrasound processing apparatus for providing electrical signals generated by the ultrasound transducer array to the ultrasound processing apparatus and receiving electrical signals generated by the ultrasound processing apparatus, wherein the plurality of first flexible electrical conductors extend throughout the entire length of the first flexible cable and the ultrasound connector is the contact section. Consequently, by using a single flexible printed circuit for the entire electrical connection between the ultrasound array and the ultrasound connector it becomes simpler to manufacture the endoscope.

In some embodiments according to a second aspect, the plurality of first flexible electrical conductors are folded at least once to extend their length. Consequently, the cost of manufacturing the flexible printed circuit may be reduced.

The plurality of first flexible electrical conductors may be bent around the support structure at more curve sections than the first curve section and the second curve section. As an example, the plurality of first flexible electrical conductors may be bend around the support structure at least at three, four, six, or eight curve sections.

The plurality of first flexible electrical conductors may be bent around the support structure at more curve sections than the first curve section and the second curve section. As an example, the plurality of first flexible electrical conductors may be bend around the support structure at least at three, four, six, or eight curve sections.

In some embodiments according to a second aspect, the support structure has a first side surface arranged in a plane being perpendicular to the first axis, the first side surface being connected to the convex upper surface and wherein the first curve section is arranged along the connection between the convex upper surface and the first side surface.

In some embodiments according to a second aspect, the second curve section is arranged along the connection between the convex upper surface and the first side surface and divided from the first curve section by a gap. Consequently, it may become possible to guide a significant number (or all) of the flexible electrical conductors away from the convex upper surface of the support structure in a simple and compact manner.

In some embodiments according to a second aspect, the support structure comprises a concave lower surface opposite to the convex upper surface, the first side surface is connected to the concave lower surface, the gap extends along the entire first side of the support structure, and wherein the plurality of the first flexible electrical conductors are further bent around the support structure at a third curve section and at a fourth curve section, the third curve section being separate from the fourth curve section by the gap, and wherein the third curve section and the fourth curve section are arranged along the connection between the first side surface and the concave lower surface. Consequently, the concave lower surface may be utilized to provide space for directing the plurality of first flexible electrical conductors away from the ultrasound transducer elements. This may reduce the amount of space required by the electric circuit.

According to a third aspect, the present disclosure relates to a curvilinear ultrasound transducer for use in connection with an endoscopic procedure. In various embodiments, the curvilinear ultrasound transducer comprises the printed flexible electrical circuit of the second aspect. In various embodiments, the curvilinear ultrasound transducer is made by the method according to the first aspect.

In one embodiment according to the third aspect, the curvilinear ultrasound transducer comprises an ultrasound transducer array comprising a plurality of ultrasound transducer elements made of a piezoelectric material, a support structure having a curvilinear upper surface, and one or more acoustic matching layers, the ultrasound transducer array being arranged above the curvilinear upper surface of the support structure, the one or more acoustic matching layers being arranged above the ultrasound transducer array and wherein a gas-filled gap is provided between each of the plurality of ultrasound transducer elements resulting in a plurality of gas-filled gaps. Consequently, by having gas-filled gaps between the ultrasound transducer elements, it may become easier to manufacture a curvilinear ultrasound transducer, as the piezoelectric material may be shaped curvilinear with layers such as an acoustic matching layer arranged on top. The ultrasound transducer may be made by the embodiments of the method according to the first aspect.

The ultrasound transducer may be provided with one or more acoustic matching layers configured to provide an acoustic impedance gradient between the acoustic impedance of the tissue and the acoustic impedance of the ultrasound transducer elements. An acoustic matching layer may be made of epoxy, polyurethane, or polystyrene.

The ultrasound transducer may be configured to generate ultrasound with a centre frequency between 1 MHz and 15 MHz, or 2 MHz and 8 MHz.

The plurality of ultrasound transducer elements may be arranged in a common reference plane and configured to image a single image plane.

In some variations of the embodiments according to the third aspect, the ultrasound transducer further comprises an acoustic lens, the acoustic lens being formed as a single element and being arranged above the first acoustic matching layer. The acoustic lens may mechanically stabilize the curvilinear ultrasound transducer with the gas-filed gaps.

In some variations of the embodiments according to the third aspect, each ultrasound transducer element of the plurality of ultrasounds transducer elements is provided with an individual acoustic matching layer. Accordingly, transfer of acoustic energy between transducer elements may be limited.

In some variations of the embodiments according to the third aspect, the curvilinear ultrasound transducer further comprises a common acoustic matching layer arranged above the ultrasound transducer array. Consequently, the common acoustic matching layer may mechanically stabilize the curvilinear ultrasound transducer with the gas-filed gaps.

In some variations of the embodiments according to the third aspect, the curvilinear ultrasound transducer further comprises a flexible electrical circuit comprising a plurality of first electrical contacts each first electrical contact being connected to an ultrasound transducer element for providing and receiving electrical signals to the ultrasound transducer element, the flexible electrical circuit being arranged between the curvilinear upper surface of the support structure and the plurality of ultrasound transducer elements.

In another embodiment according to the third aspect, the present disclosure relates to an ultrasound transducer for receiving and transmitting ultrasound comprising a convex ultrasound transducer array comprising a plurality of ultrasound transducer elements, an electrical circuit for receiving and providing electrical signals to the plurality of ultrasound transducer elements, and a support structure having a convex upper surface facing the plurality of ultrasound transducer elements and extending in a direction parallel to a first axis, wherein the electrical circuit comprises: a flexible base section comprising a plurality of first electrical contacts each first electrical contact being connected to an ultrasound transducer element for providing and receiving electrical signals to/from the ultrasound transducer element and extending along a central axis being parallel to the first axis, the flexible base section being arranged on the convex upper surface of the support structure; a plurality of first flexible electrical conductors each first flexible electrical conductor being electrically connected to a first electrical contact; a contact section comprising a plurality of second electrical contacts each second electrical contact being electrically connected to a first electrical conductor and further electrical connected or connectable to a second electrical conductor or a transducer port of an ultrasound processing apparatus, wherein the plurality of first flexible electrical conductors are bent around the support structure at a first curve section and at a second curve section, the first curve section being separate from the second curve section.

Consequently, by using an electrical circuit provided with a flexible base section having a plurality of electrical contacts, the plurality of ultrasound transducer elements may be provided with an electrical connection in a single process step. This makes it simple to manufacture. Additionally, by providing the electrical circuit with a plurality of flexible electrical conductors bent around the support structure at a first curve section and second curve section, a single electrical circuit may be used to transport the electrical signal to and from the plurality of electrical contacts as the electric circuit may conform to the curvature of the convex upper surface.

The flexible base section and/or the plurality of first flexible conductors may be a flexible printed circuit. The flexible printed circuit may be made using Photolithography. An adhesive may be provided between the lower side of the ultrasound transducer array and the flexible base section. Correspondingly, an adhesive may be provided between the flexible base section and the convex upper surface of the support structure. After attachment to the plurality of ultrasound transducer elements the flexibility of the flexible base section is limited by the flexibility of the ultrasound transducer array.

The ultrasound transducer elements may be made from a piezo electric block. The piezoelectric block may be made of any kind of piezoelectric material such as lead zirconate titanate (PZT), Quartz, Barium titanate, Poly(vinylidene fluoride) (PVDF), or a piezoelectric composite material.

The ultrasound transducer may be provided with one or more acoustic matching layers configured to provide an acoustic impedance gradient between the acoustic impedance of the tissue and the acoustic impedance of the ultrasound transducer elements. The acoustic matching layer may be made of epoxy, polyurethane, or polystyrene.

The ultrasound transducer may be configured to generate ultrasound with a center frequency between 1 MHz and 15 MHz, or 2 MHz and 8 MHz.

The plurality of ultrasound transducer elements may be arranged in a common reference plane and configured to image a single image plane.

The plurality of ultrasound transducer elements may have been pre-poled and metalized before being attached to flexible base section. As an example, one or two layers of a nickel-chromium alloy may be applied to the plurality of ultrasound transducer elements. Alternatively/additionally one or two layers of Au may be applied. The layer may preferably be thin layers having a thickness of 10-20 nm. The layers may improve the piezoelectric properties of the piezoelectric block.

The support structure is preferably made of a material configured to absorb ultrasound energy. The material may be a composite material. As an example, the material may be an epoxy mixed with tungsten powder.

The flexible base section, the plurality of first flexible electrical conductors, and the contact section of the electrical circuit may be produced as a single integral element.

The ultrasound transducer may further comprise an acoustic lens for focusing the ultrasound energy. The acoustic lens may be configured to focus the ultrasound energy in the image plane.

The plurality of first flexible electrical conductors may be bent around the support structure at more curve sections than the first curve section and the second curve section. As an example, the plurality of first flexible electrical conductors may be bent around the support structure at least at three, four, six, or eight curve sections.

In some variations of the present embodiment, the support structure has a first side surface arranged in a plane being perpendicular to the first axis, the first side surface being connected to the convex upper surface and wherein the first curve section is arranged along the connection between the convex upper surface and the first side surface.

In some variations of the present embodiment, the second curve section is arranged along the connection between the convex upper surface and the first side surface and divided from the first curve section by a gap. Consequently, it may become possible to guide a significant number (or all) of the flexible electrical conductors away from the convex upper surface of the support structure in a simple and compact manner.

In some variations of the present embodiment, the support structure comprises a concave lower surface opposite to the convex upper surface, the first side surface is connected to the concave lower surface, the gap extends along the entire first side of the support structure, and wherein the plurality of the first flexible electrical conductors are further bent around the support structure at a third curve section and at a fourth curve section, the third curve section being separate from the fourth curve section by the gap, and wherein the third curve section and the fourth curve section are arranged along the connection between the first side surface and the concave lower surface. Consequently, the concave lower surface may be utilized to provide space for directing the plurality of first flexible electrical conductors away from the ultrasound transducer elements. This may reduce the amount of space required by the electric circuit.

In some variations of the present embodiment, the plurality of first flexible electrical conductors comprises a first group of flexible electrical conductors and a second group of flexible electrical conductors, the support structure has a second side surface opposite to the first side surface, the second side surface arranged in a plane being perpendicular to the first axis, the second side surface being connected to the convex upper surface, the second curve section is arranged along the connection between the convex upper surface and the second side surface, and wherein the first group of flexible electrical conductors are bent around the first curve section and the second group of flexible electrical conductors are bent around the second curve section. Consequently, it may become possible to utilize space on both sides of the support structure for the plurality of first electrical conductors.

In some variations of the present embodiment, the first group of flexible electrical conductors are arranged in a first plane along a part of their length, the second group of flexible electrical conductors are arranged in a second plane along a part of their length, where the first plane is arranged above the second plane. Consequently, by packing the flexible electrical conductors vertically, the required space in the width may be reduced. This may be advantageous as flexible electrical may be created very thin, whereby the increased space required in the height may handle relatively easily.

In some variations of the present embodiment, the first plane is parallel with the second plane.

In some variations of the present embodiment, each of the plurality of second electrical contacts are connected to a second electrical conductor.

The first electrical circuit will typically be manufactured as a flat structure and subsequently folded into the form required by the ultrasound transducer. Thus, by connecting the second electrical contact to a second electrical conductor the folding task may be simplified.

In some variations of the present embodiment, for each of the plurality of second electrical contacts the second electrical conductor is a coaxial cable permanently connected to the second electrical contact. Consequently, by using coaxial cables in connection with a flexible electrical circuit the connections between the coaxial cables and the ultrasound transducer elements may be done at a distance from the ultrasound transducer elements, where there is more space.

In some variations of the present embodiment, for each of the plurality of second electrical contacts the second electrical conductor is a second flexible electrical conductor permanently connected the second electrical contact. Consequently, by also using flexible electrical conductors to transfer electrical signal further away from the transducer elements, the space requirements of the entire electrical connection may be kept low.

In some variations of the present embodiment, each of the plurality of second electrical contacts are configured to be detachable connectable to a second electrical conductor or a transducer port of an ultrasound processing apparatus.

In some variations of the present embodiment, the flexible base section is a single flexible printed circuit. Consequently, both the manufacturing of the base section and the subsequent attachment to the ultrasound transducer elements may be simplified.

In some variations of the present embodiment, the flexible base section and the plurality of first flexible electrical conductors is a single flexible printed circuit. Consequently, both the manufacturing of the base section and the subsequent attachment to the ultrasound transducer elements may be simplified.

According to a fourth aspect, the present disclosure relates to an endoscope comprising a handle, an insertion tube, an image capturing device (or image sensor), a first flexible cable and an ultrasound transducer as disclosed in relation to the third aspect, the handle being attached to a proximal end of the insertion tube, the image capturing device and the ultrasound transducer being attached to a distal end of the insertion tube, and the first flexible cable extending from the handle and being electrically connected to the plurality of ultrasound transducer elements via the electrical circuit according to the second aspect, wherein the first flexible cable is connectable to an ultrasound processing apparatus and configured to provide the ultrasound processing apparatus with electrical signals generate by the plurality of ultrasound transducer elements and receive electrical signals generated by the ultrasound processing apparatus for the plurality of ultrasound transducer elements.

Ultrasound processing apparatuses, or ultrasound transducer controllers, are well known in the art. Ultrasound processing apparatuses are configured to generate the electrical signals needed by the ultrasound transducer elements to generate the ultrasound waves. The electrical signal should have a center frequency substantially matching the center frequency of the ultrasound transducer elements. Furthermore, the signal should have a length and amplitude securing that a sufficient signal-to-noise ratio (SNR) is achieved. Furthermore, the ultrasound processing apparatus is configured to generate the ultrasound images by processing the signals generated by the ultrasound transducer elements. The ultrasound imaging may be done by using ultrasound beamforming techniques such as delay and sum beamforming in receive. In transmit, the ultrasound energy may be focused in a particular point by applying a suitable delay to the individual ultrasound transducer elements. Alternatively, a plane wave or a diverging ultrasound wave may be used in transmit.

The ultrasound processing apparatus may be adapted to cause a display to display a live representation of the electrical signals received from the ultrasound transducer. The ultrasound processing apparatus may be couplable to a display. The ultrasound processing apparatus may comprise a display for displaying a live representation of the electrical signals received from the ultrasound transducer and/or the image capturing device.

The ultrasound processing apparatus may be a portable ultrasound processing apparatus.

In some embodiments, the first flexible cable is further configured to receive electrical signals from the image capturing device and provide the received electrical signals to the ultrasound processing apparatus. Consequently, by using a single flexible cable to transfer signals from both the ultrasound transducer and the image capturing device it may become simpler for user to operate the endoscope.

The ultrasound processing apparatus may comprise one or more processing units configured to process both the signals received from the ultrasound transducer and the signals received from the image capturing device.

Alternatively, the ultrasound processing apparatus may comprise a first set of one or more processing units configured to process the signals received from the ultrasound transducer and a second set of one or more processing units configured to process the signals received form the image capturing device. The first set and second set of one or more processing units may be provided in the same housing or in separate housings where the separate housings are communicatively coupled.

In some embodiments, the endoscope further comprises a second flexible cable configured to receive electrical signals from the image capturing device and provide the received electrical signals to a video processing apparatus. Consequently, it may become simpler to connect the endoscope to a separate ultrasound processing apparatus and video processing apparatus.

The video processing apparatus may be adapted to cause a display to display a live representation of the electrical signals received from image capturing device. The video processing apparatus may be couplable to a display. The video processing apparatus may comprise a display for displaying a live representation of the electrical signals received from the image capturing device.

The video processing apparatus may be a portable video processing apparatus.

In some embodiments, the first flexible cable and the second flexible cable are connected along a first part of their length and disconnected along a second part of their length. Consequently, it may both become simpler for the user to operate the endoscope and connect the cables to a separate ultrasound processing apparatus and video processing apparatus.

The first flexible cable may be attached to the second cable using an adhesive. Additionally/alternatively an outer cable may be arranged around the first flexible cable and the second flexible cable along the first part of their length.

According to a fifth aspect, the present disclosure relates to a system comprising an endoscope as disclosed in relation to the fourth aspect of the present disclosure and an ultrasound processing apparatus, wherein the first flexible cable is connectable to the ultrasound processing apparatus.

In some embodiments according to the fifth aspect, the first flexible cable is further configured to receive electrical signals from the image capturing device and provide the received electrical signals to the ultrasound processing apparatus, and wherein the ultrasound processing apparatus comprise one or more processing units configured to process both the signals received from the ultrasound transducer and the signals received from the image capturing device.

In some embodiments according to the fifth aspect, the system further comprises a video processing apparatus and wherein the endoscope further comprises a second flexible cable configured to receive electrical signals from the image capturing device and provide the received electrical signals to the video processing apparatus.

In some embodiments according to the fifth aspect, the first flexible cable and the second flexible cable are connected along a first part of their length and disconnected along a second part of their length.

The different aspects of the present disclosure can be implemented in different ways including as an ultrasound transducer, a printed flexible electrical circuit, an endoscope comprising an ultrasound transducer, and an endoscope system described above and in the following, each yielding one or more of the benefits and advantages described in connection with at least one of the aspects described above, and each having one or more preferred embodiments corresponding to the preferred embodiments described in connection with at least one of the aspects described above and/or disclosed in the dependent claims. Furthermore, it will be appreciated that embodiments described in connection with one of the aspects described herein may equally be applied to the other aspects.

The different aspects of the present disclosure can be implemented in different ways including methods of manufacturing a curvilinear ultrasound transducer, a curvilinear ultrasound transducer and an endoscope comprising a curvilinear ultrasound transducer described above and in the following, each yielding one or more of the benefits and advantages described in connection with at least one of the aspects described above, and each having one or more preferred embodiments corresponding to the preferred embodiments described in connection with at least one of the aspects described above and/or disclosed in the dependent claims. Furthermore, it will be appreciated that embodiments described in connection with one of the aspects described herein may equally be applied to the other aspects.

### BRIEF DESCRIPTION OF THE DRAWINGS

The above described and/or additional objects, embodiments, variations thereof, examples, features and advantages of the present disclosure will be elucidated with reference to the following non-limiting drawings.

FIG. 1 shows a method of manufacturing a curvilinear ultrasound transducer according to an embodiment of the disclosure;

FIGS. 2A and 2B show schematically a support structure for an ultrasound transducer according to an embodiment of the disclosure;

FIGS. 3A, 3B and 3C show schematically a support structure and an electrical circuit for an ultrasound transducer according to an embodiment of the disclosure;

FIGS. 4A, 4B and 4C show schematically a support structure and an electrical circuit for an ultrasound transducer according to an embodiment of the disclosure;

FIGS. 5A and 5B show schematically a support structure and an electrical circuit for an ultrasound transducer according to an embodiment of the disclosure;

FIG. 6 shows schematically an ultrasound transducer according to an embodiment of the disclosure;

FIG. 7 shows a method of manufacturing a curvilinear ultrasound transducer according to an embodiment of the disclosure;

FIGS. 8A and 8B show a method of manufacturing a curvilinear ultrasound transducer according to an embodiment of the disclosure;

FIG. 9 shows a method of manufacturing a curvilinear ultrasound transducer according to an embodiment of the disclosure;

FIGS. 10A and 10B show schematically an embodiment of an endoscope according to the disclosure;

FIG. 11 shows schematically a system according to an embodiment of the disclosure; and

FIG. 12 shows schematically a system according to an embodiment of the disclosure.

### DETAILED DESCRIPTION

In the following description, reference is made to the accompanying figures, which show by way of illustration how the embodiments of the present disclosure may be practiced.

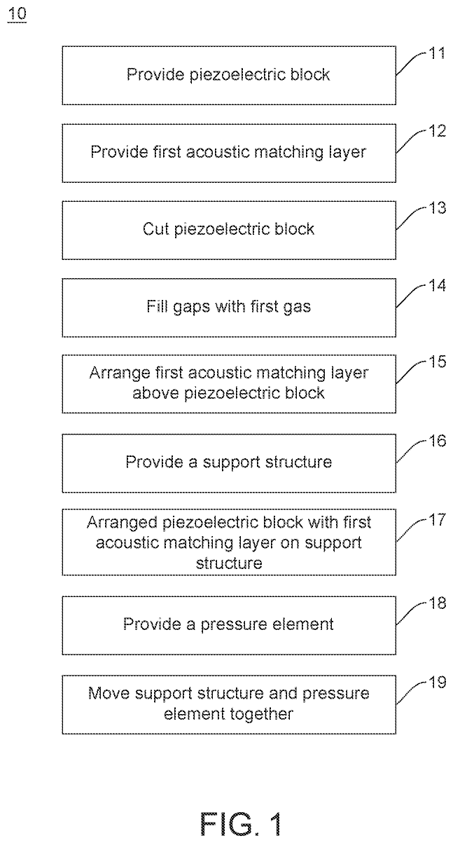

FIG. 1 shows a method 10 of manufacturing a curvilinear ultrasound transducer for use with an endoscope according to an embodiment of the disclosure. The method begins at 11, wherein a piezoelectric block having an upper side and a lower side opposite to the upper side is provided. At 12, a first acoustic matching layer is provided. At step 13, the piezoelectric block is cut at a plurality of predetermined positions forming a plurality of ultrasound transducer elements separated by a plurality of gaps. At 14, the plurality of gaps are allowed to be filed with a first gas resulting in a plurality of gas-filled gaps. At 15, the first acoustic matching layer is arranged above the piezoelectric block, optionally after the piezoelectric block has been cut. At 16, a support structure is provided having a curvilinear upper surface. At 17, the piezoelectric block with the first acoustic matching layer is arranged above the curvilinear upper surface of the support structure, optionally after the piezoelectric block has been cut. At 18, a pressure element is provided having a concave surface. At 19, the support structure and the pressure element is moved together to shape the piezoelectric block curvilinear.

While the different steps of the method are shown in a particular order, it should be understood that the steps may be performed in a number of different orders. As an example, the cutting, at 13, may be performed after the first acoustic matching layer is arranged above the piezoelectric block, at 15, but before the piezoelectric block has been shaped curvilinear, at 19. The cutting, at 13, may also be performed at the end, i.e. after the piezoelectric block has been shaped curvilinear, at 19.

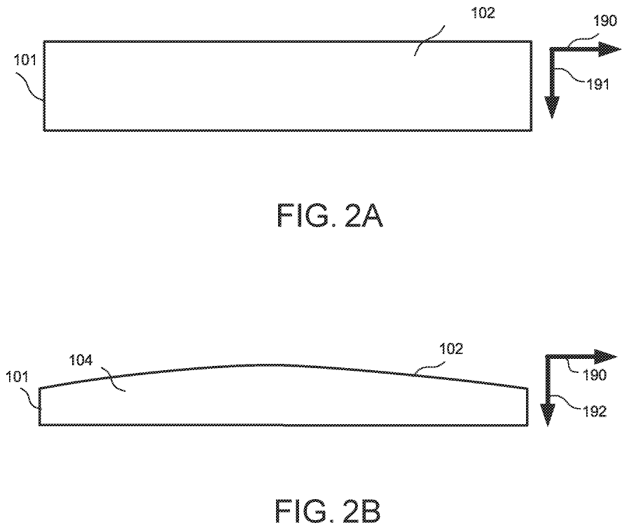

FIGS. 2A and 2B show schematically a support structure (101) for an ultrasound transducer according to an embodiment of the disclosure, where FIG. 2A shows a top view and FIG. 2B shows a side view. The ultrasound transducer may be manufactured by according to the embodiments of the method of the first aspect. Referring to FIGS. 2A and 2B, the support structure (101) has a convex upper surface (102) for facing a plurality of ultrasound transducer elements. The convex upper surface (102) extends in a direction parallel to a first axis (191). There is further shown a second axis (190) and a third axis (192). The second axis (190) being perpendicular to the first axis (191), and the third axis (192) being perpendicular to both the first axis (191) and the second axis (190). In addition to supporting the ultrasound transducer elements, the support structure may assist in absorbing ultrasound energy. Thus, the support structure (101) is preferably made of a material configured to absorb ultrasound energy. The material may be a composite material. The material may be an epoxy mixed with tungsten powder.

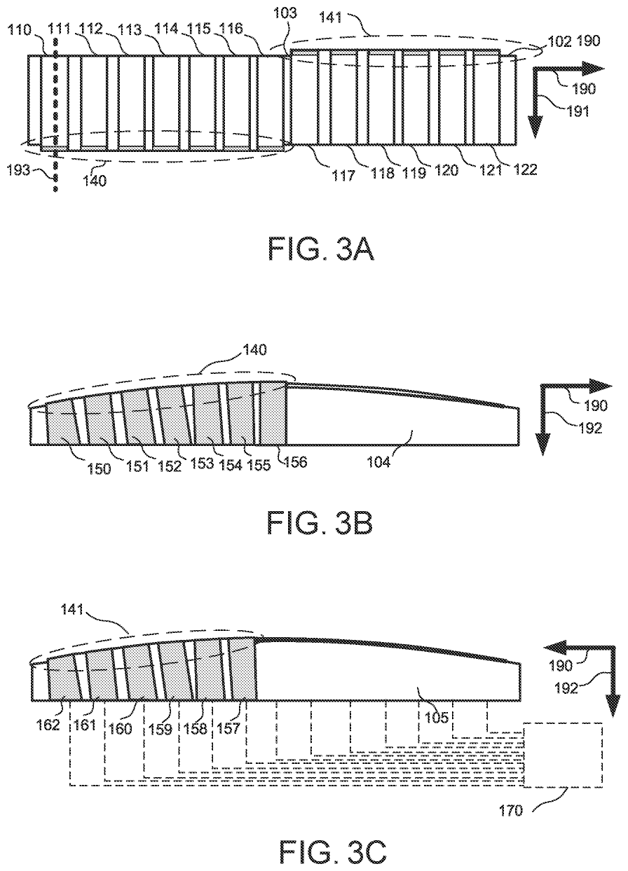

FIGS. 3A-C show schematically a support structure (101) and an electrical circuit for an ultrasound transducer according to an embodiment of the disclosure, where FIG. 2a shows a top view, FIG. 3A shows a first side view, and FIG. 3C shows a second side view. The support structure (101) corresponds to the support structure (101) shown in FIG. 2A and 2B. The electrical circuit comprises a flexible base section (103) comprising a plurality of first electrical contacts (110-122) each first electrical contact (110-122) being connectable to an ultrasound transducer element for providing and receiving electrical signals to/from the ultrasound transducer element.

Each of the first electrical contacts (110-122) extends along a central axis (193) being parallel to the first axis (191). For simplicity only the central axis (193) for the first electrical contact 110 is shown. The flexible base section (103) is arranged on the convex upper surface (102) of the support structure (101). In the shown example the flexible base section (103) covers substantially the whole surface area of the convex upper surface (102). The first electrical contacts (110-122) should be made of a material having a high electrical conductivity such as Cu, Al, Ag, or Au. The area of the flexible base section (103) between the plurality of first electrical contacts (110-122) should be electrically isolating to prevent crosstalk between the ultrasound transducer elements. Correspondingly, the flexible base section (103) is preferably provided with one or more electrically isolating lower layers. For simplicity only 13 first electrical contacts (110-122) are shown. However, in other embodiments there may be at least 16, 32 or 64 first electrical contacts. The electrical circuit further comprises a plurality of first flexible electrical conductors (150-162) each first flexible electrical conductor being electrically connected to a first electrical contact (110-122).

The plurality of first flexible electrical conductors (150-162) are responsible for transferring electrical signals from the plurality of first electrical contacts (110-122) towards an ultrasound processing apparatus and transferring electrical signals from an ultrasound processing apparatus towards the plurality of first electrical contacts (110-122). The plurality of first flexible electrical conductors (150-162) should be made of a material having a high electrical conductivity such as Cu, Al, Ag, or Au. The area between the plurality of first flexible electrical conductors (150-162) should be electrically isolating to prevent cross-talk between the ultrasound transducer elements. Correspondingly, the plurality of first flexible electrical conductors (150-162) are preferably provided with one or more electrically isolating upper layers and one or more electrically isolating lower layers.

The plurality of first flexible electrical conductors (150-162) are bent around the support structure (101) at a first curve section (140) and at a second curve section (141), the first curve section (140) being separate from the second curve section (141). The plurality of first flexible electrical conductors comprises a first group of flexible electrical conductors (150-156) and a second group of flexible electrical conductors (157-162). The support structure (101) has a first side surface (104) being arranged in a plane (not shown) being perpendicular to the first axis (191), the first side surface (104) being connected to the convex upper surface (102) and wherein the first curve section (140) is arranged along the connection between the convex upper surface (102) and the first side surface (104). The support structure (101) has a second side surface (105) opposite to the first side surface (104), the second side surface (105) being arranged in a plane (not shown) being perpendicular to the first axis (191), the second side surface (105) being connected to the convex upper surface (102), the second curve section (141) is arranged along the connection between the convex upper surface (102) and the second side surface (105). The first group of flexible electrical conductors (150-156) are bent around the first curve section (140) and the second group of flexible electrical conductors (157-162) are bent around the second curve section (141). Only the first part of the plurality of first electrical conductors is shown in FIGS. 3A-3C. In FIG. 3C are the remaining part of the first electrical conductors are schematically illustrated by dashed lines. The electrical circuit further comprises a contact section (170) comprising a plurality of second electrical contacts each second electrical contact being electrically connected to a first electrical conductor (150-162) and further electrical connected or connectable to a second electrical conductor or a transducer port of an ultrasound system.

In some embodiments, the contact section (170) is the ultrasound connector that is inserted into the transducer port of an ultrasound processing apparatus and the first electrical conductors (150-162) are responsible for the entire electrical coupling between the plurality of first electrical contacts (110-122) and the ultrasound connector.

In other embodiments, each of the plurality of second electrical contacts of the contact section (170) are connected to a second electrical conductor and the first electrical conductors (150-162) are only responsible for part of the electrical coupling between the plurality of first electrical contacts (110-122) and the ultrasound connector. The second electrical conductor may be a coaxial cable or a printed flexible electrical conductor.

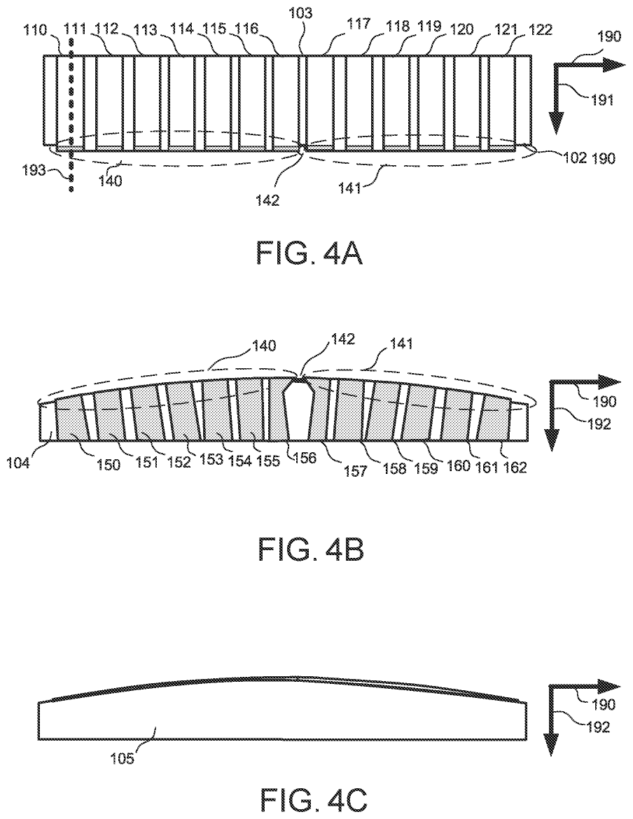

FIGS. 4A-C show schematically a support structure (101) and an electrical circuit for an ultrasound transducer according to an embodiment of the disclosure, where FIG. 4A shows a top view, FIG. 4B shows a first side view, and FIG. 4C shows a second side view. The support structure (101) corresponds to the support structure (101) shown in FIG. 2A and 2B. The electrical circuit comprises a flexible base section (103) comprising a plurality of first electrical contacts (110-122) each first electrical contact (110-122) being connectable to an ultrasound transducer element for providing and receiving electrical signals to/from the ultrasound transducer element. Each of the first electrical contacts (110-122) extends along a central axis (193) being parallel to the first axis (191). For simplicity only the central axis (193) for the first electrical contact 110 is shown. The flexible base section (103) is arranged on the convex upper surface (102) of the support structure (101). The electrical circuit further comprises a plurality of first flexible electrical conductors (150-162) each first flexible electrical conductor being electrically connected to a first electrical contact (110-122). The plurality of first flexible electrical conductors (150-162) are responsible for transferring electrical signals from the plurality of first electrical contacts (110-122) towards an ultrasound processing apparatus and transferring electrical signals from an ultrasound processing apparatus towards the plurality of first electrical contacts (110-122). The support structure (101) has a first side surface (104) arranged in a plane (not shown) being perpendicular to the first axis (191), the first side surface (104) being connected to the convex upper surface (102). The plurality of first flexible electrical conductors (150-162) are bent around the support structure (101) at a first curve section (140) and at a second curve section (141), the first curve section (140) being separate from the second curve section (141). The first curve section (140) and the second curve section (141) are arranged along the connection between the convex upper surface (102) and the first side surface (104) and divided by a gap (142). Only the first part of the plurality of first electrical conductors is shown in FIGS. 4A-4C. The electrical circuit may further comprise a contact section (170) comprising a plurality of second electrical contacts each second electrical contact being electrically connected to a first electrical conductor (150-162) and further electrical connected or connectable to a second electrical conductor or a transducer port of an ultrasound system.

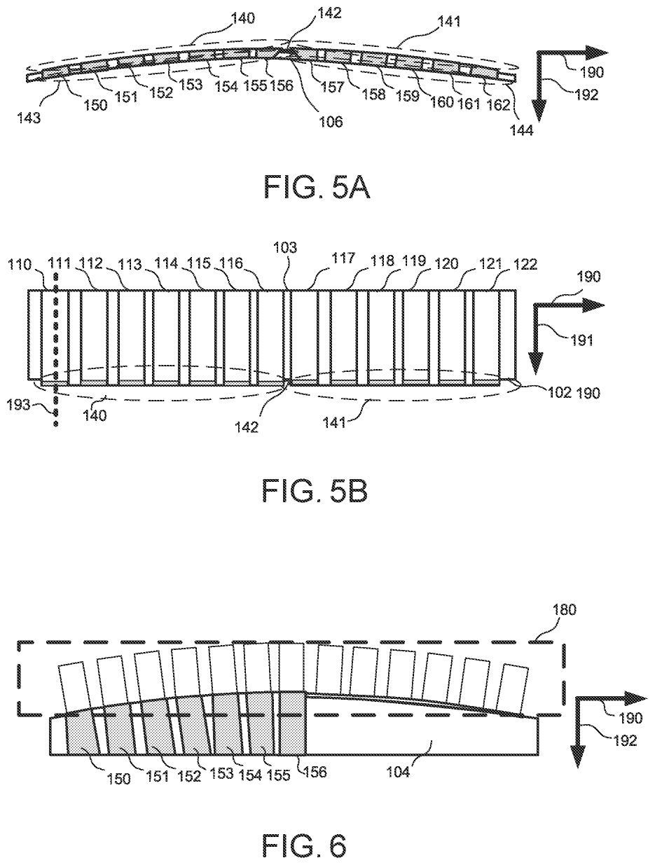

FIG. 5A-5C show schematically a support structure (101) and an electrical circuit for an ultrasound transducer according to an embodiment of the disclosure, where FIG. 5A shows a top view and FIG. 5B shows a side view. The embodiment shown in FIGS. 5A-5C corresponds to the embodiment shown in FIG. 4A-4C with the difference that the support structure (101) comprises a concave lower surface (106) opposite to the convex upper surface (102), the first side surface (104) is connected to the concave lower surface (106) and the gap (142) extends along the entire first side (104) of the support structure (101). The plurality of first flexible electrical conductors (150-162) are further bent around the support structure at a third curve section (143) and at a fourth curve section (144), the third curve section (143) being separate from the fourth curve section (144) by the gap (142), and wherein the third curve section (143) and the fourth curve section (144) are arranged along the connection between the first side surface (104) and the concave lower surface (106). Consequently, the concave lower surface may be utilized to provide space for directing the plurality of first flexible electrical conductors away from the ultrasound transducer elements. This may reduce the amount of space required by the electric circuit.

FIG. 6 shows schematically an ultrasound transducer according to an embodiment of the disclosure. The ultrasound transducer comprises a support structure (101) and an electrical circuit corresponding to the support structure and the electrical circuit disclosed in relation to FIGS. 3A-3C. Furthermore, the ultrasound transducer comprises a convex ultrasound transducer array comprising a plurality of ultrasound transducer elements (180), where each first electrical contact (110-122) is connected to an ultrasound transducer element for providing and receiving electrical signals to/from the ultrasound transducer element.

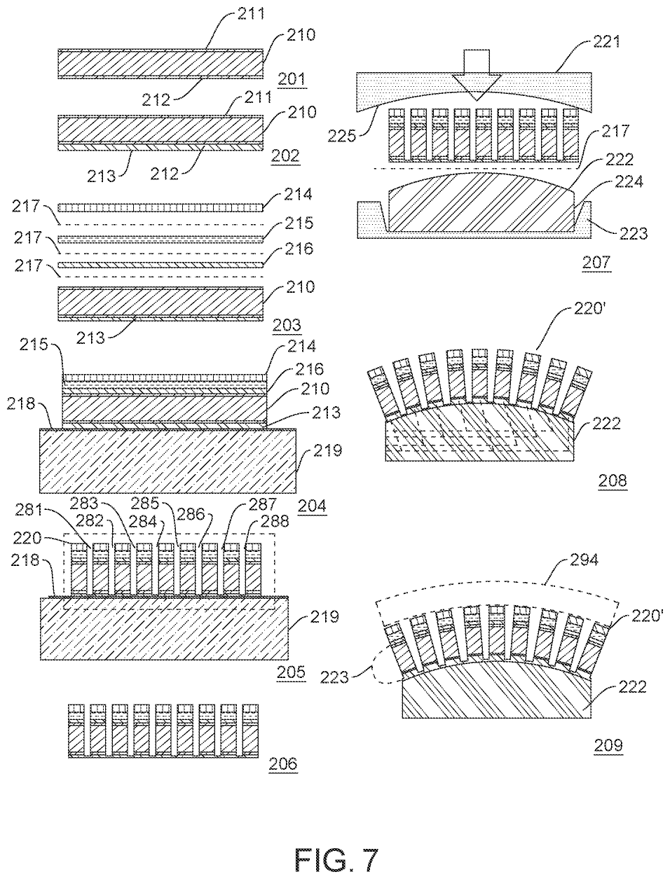

FIG. 7 shows a central cross-section at various stages of an embodiment of a method of manufacturing a curvilinear ultrasound transducer for use with an endoscope. In step 201, a piezoelectric block 210 is provided having an upper side and a lower side opposite to the upper side. A metal electrode 211 is provided on the upper side and a metal electrode 212 is provided on the lower side of the piezoelectric block 210. The piezoelectric block 210 may previously have been pre-poled and metalized. As an example, one or two layers of a nickel-chromium alloy may be applied to the piezoelectric block. Alternatively/additionally one or two layers of Au may be applied. The layer may preferably be thin layers having a thickness of 10-20 nm. The layers may improve the piezoelectric properties of the piezoelectric block.

Next in step 202, a flexible electrical circuit 213 is provided for electrically receiving and providing electrical signals to a plurality of ultrasound transducer elements.

The flexible electrical circuit 213 comprises a plurality of first electrical contacts, where each first electrical contact is connectable to an ultrasound transducer element for providing and receiving electrical signals to the ultrasound transducer element. The flexible electrical circuit 213 is attached to the lower side of the piezoelectric block 210. The flexible electrical circuit 213 may be glued onto the lower side of the piezoelectric block 210.

Next in step 203, a common ground electrode 216 is provided and arranged above the piezoelectric block 210. A layer of adhesive 217 is provided between the common ground electrode 216 and the piezoelectric block 210. Additionally, a first acoustic matching layer 215 and a second acoustic matching layer 214 is provided. The first acoustic matching layer 215 is arranged above the piezoelectric block 210 and attached to the common ground electrode 216 using an adhesive 217. The second acoustic matching layer 214 is arranged above the piezoelectric block 210 and attached to the first acoustic matching layer 215 using an adhesive 217.

Then in step 204, the piezoelectric block 210 is arranged on a temporary support 219 to prepare the piezoelectric block 210 for cutting. The piezoelectric block 210 may be attached to the temporary support 219 using a release tape 218. The release tape may be a thermal release tape configured to adhere tightly to the piezoelectric block at a first temperature such as room temperature and release the piezoelectric block 210 at a second temperature such as a slightly elevated temperature. Alternatively, the release tape may be a UV release tape configured to adhere tightly to the piezoelectric block 210 before being exposed to UV radiation and release the piezoelectric block 210 after having been expose to UV radiation. If UV release tape is being used, the temporary support 219 is preferably made of a material permeable to UV radiation e.g. the temporary support 219 may be made of a glass material permeable to UV radiation.

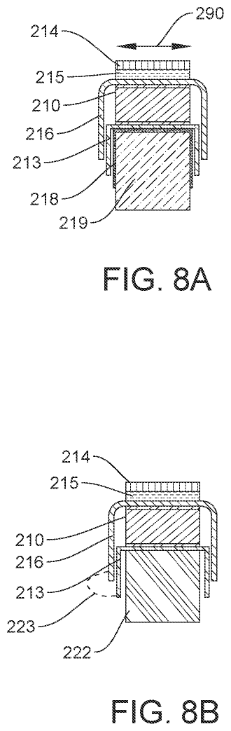

Next in step 205, the piezoelectric block 210 is cut at a plurality of predetermined positions thereby forming a plurality of ultrasound transducer elements 220 separated by a plurality of gaps. FIG. 8A shows a side view of the assembly in the state of step 205, i.e. after cutting. Preferably, the cutting/dicing blade may have a width corresponding the width of the plurality of gaps. The gaps are also known as the kerf of the ultrasound transducer. In the shown embodiment, 9 ultrasound transducer elements are shown. However, typically more ultrasound transducer elements are formed e.g. at least 16, at least 32 or at least 64 ultrasound transducer elements. The cutting is done through the first acoustic matching layer 215, the second acoustic matching layer 214, (parts of) the common ground electrode 216 and the piezoelectric block 210. Preferably, the cutting is done through the entire piezoelectric block 210 but without significantly cutting into flexible electrical circuit 213. The common ground electrode 216 has a width being wider than the width 290 of the upper side of the piezoelectric block (as seen in FIG. 8A), whereby the cutting of the piezoelectric block can be done while keeping all parts of the common ground electrode 216 electrically connected. After cutting the plurality of gaps is allowed to be filed with a first gas resulting in a plurality of gas-filled gaps 281-288.

Then, in step 206 the plurality of ultrasound transducer elements 220 are released from the release tape 218 and the temporary support 219.

Next in step 207, a support structure 224 having a curvilinear upper surface 222 is provided. The piezoelectric block with the first acoustic matching layer 215, the second acoustic matching layer 214, and the common ground electrode 216 is arranged above the curvilinear upper surface of the support structure 222 with a layer of adhesive 217 provided between the piezoelectric block and the support structure 222. In this embodiment, this is done after the piezoelectric block has been cut. Additionally, a pressure element 221 is provided having a concave surface 225, and a support 223 for supporting the support structure 224 in step 207.

Then, the support structure 224 and the pressure element 221 is moved together to shape the piezoelectric block curvilinear 220′ as shown in step 208.

Next, in step 209 an electrical connection 223 is provided between common ground electrode 216 and the flexible electrical circuit 213. FIG. 8B shows a side view of the assembly in step 209. Finally, an acoustic lens 294 may be arranged above the first and second acoustic matching layer 215, 216.

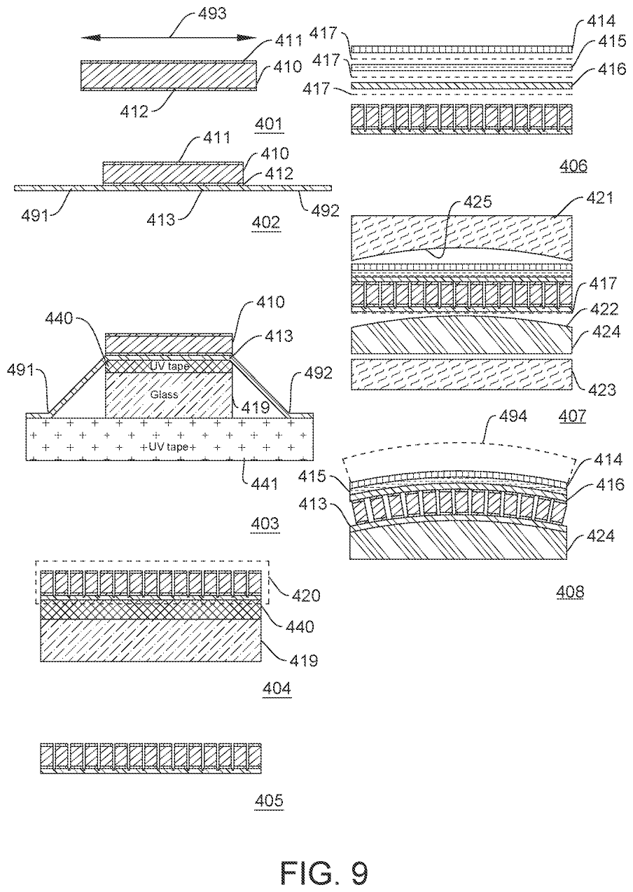

FIG. 9 shows a central cross-section at various stages of another embodiment of a method of manufacturing a curvilinear ultrasound transducer for use with an endoscope. In step 401 a piezoelectric block 410 is provided having an upper side and a lower side opposite to the upper side. A metal electrode 411 is provided on the upper side and a metal electrode 412 is provided on the lower side of the piezoelectric block 410. The piezoelectric block 410 may previously have been pre-poled and metalized.

Next in step 402, a flexible electrical circuit 413 is provided for electrically receiving and providing electrical signals to a plurality of ultrasound transducer elements. The flexible electrical circuit 413 comprises a plurality of first electrical contacts, where each first electrical contact is connectable to an ultrasound transducer element for providing and receiving electrical signals to the ultrasound transducer element. The flexible electrical circuit 413 is attached to the lower side of the piezoelectric block 410. The flexible electrical circuit 413 may be attached using an adhesive (not shown) to the lower side of the piezoelectric block 410. In this embodiment, the flexible electrical circuit 413 has a first part 491 that extends beyond the length 493 of the piezoelectric block 410 and a second part 492 that extends beyond the length 493 of the piezoelectric block 410. The first part 491 and the second part 492 may be utilized for transferring the signals to and from the plurality of ultrasound transducer elements.

Then in step 404, the piezoelectric block 410 is arranged on a temporary support 419 to prepare the piezoelectric block 410 for cutting. The piezoelectric block 410 is attached to the temporary support 419 using a release tape 240. The release tape may be a thermal release tape or a UV release tape. If UV release tape is being used, the temporary support 219 is preferably made of a material permeable to UV radiation e.g. the temporary support 219 may be made of a glass material permeable to UV radiation. Additionally, the first part of the flexible electric circuit 491 and the second part of the flexible electrical circuit 492 are attached to a support using a release tape such as a UV release tape or a thermal release tape. This may secure that the flexible electrical circuit 413 is stabilized during the cutting step and further protect the first part of the flexible electric circuit 491 and the second part of the flexible electrical circuit 492.

Next in step 404, the piezoelectric block 410 is cut at a plurality of predetermined positions thereby forming a plurality of ultrasound transducer elements 420 separated by a plurality of gaps.

From step 404, the first part and second part of the flexible electrical circuit 491, 492 are no longer shown in FIG. 9; however, it should be understood that they are still present. Preferably, the cutting/dicing blade may have a width corresponding the width of the plurality of gaps. Preferably, the cutting is done through the entire piezoelectric block 410 but without significantly cutting into flexible electrical circuit 413. After cutting the plurality of gaps is allowed to be filed with a first gas resulting in a plurality of gas-filled gaps.

Then in step 405, the plurality of ultrasound transducer elements 420 are released from the release tape 418 and the temporary support 419 and the first part and second part of the flexible electrical circuit 491 492 are released from the release tape 441. Then in step 406, a common ground electrode 416 is provided and arranged above the plurality of cut ultrasound transducer elements 420. A layer of adhesive 217 is provided between the common ground electrode 213 and the piezoelectric block 210. Additionally, a first acoustic matching layer 415 and a second acoustic matching layer 414 is provided. The first acoustic matching layer 415 is arranged above the plurality of cut ultrasound transducer elements 420 and attached to the common ground electrode 416 using an adhesive 417. The second acoustic matching layer 414 is arranged above the plurality of cut ultrasound transducer elements 420 and attached to the first acoustic matching layer 415 using an adhesive 417. In this embodiment, the acoustic matching layers 415 416 are arranged above the plurality of ultrasound transducer elements 420 after the piezoelectric block 410 has been cut forming the plurality of ultrasound transducer elements 420. Consequently, the acoustic matching layers 415 414 may structurally stabilize the ultrasound transducer elements. This may also make the step of cutting the ultrasound transducer elements 420 simpler.

Next in step 407, a support structure 424 having a curvilinear upper surface 422 is provided. The piezoelectric block with the first acoustic matching layer 415, the second acoustic matching layer 414, and the common ground electrode 416 is arranged above the curvilinear upper surface of the support structure 422 with a layer of adhesive 417 provided between the piezoelectric block and the support structure 422. In this embodiment, this is done after the piezoelectric block has been cut. Additionally, a pressure element 421 is provided having a concave surface 425, and a support 423 for supporting the support structure 424 in step 407. Then the support structure 424 and the pressure element 421 are moved together to shape the piezoelectric block curvilinear as shown in step 408. Finally, an acoustic lens 494 may be arranged above the first and second acoustic matching layer 415, 416. By having the plurality of gaps filled with a first gas, it becomes simpler to manufacture a curvilinear ultrasound transducer, as the ultrasound transducer may be given the curvilinear shape with the acoustic matching layers and common ground electrode arranged on the piezoelectric block. This may reduce the number of process steps. Furthermore, the acoustic matching layers and common ground electrode may be manufactured with a planar shape, which may be significant simpler than attempting to match the specific curvilinear shape of the piezoelectric block. This may reduce the cost of manufacturing a curvilinear ultrasound transducer thereby extending the availability of endoscopic ultrasound. Furthermore, single use ultrasound endoscope may also become possible, which may eliminate risk of cross-contamination of serious illnesses.

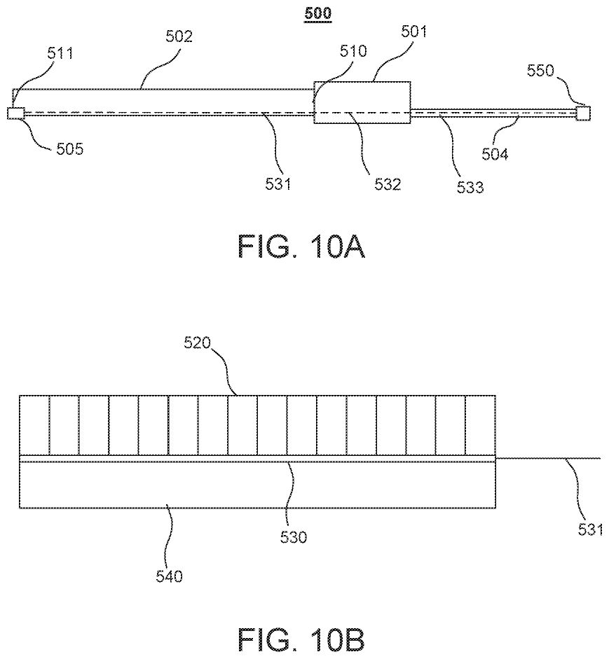

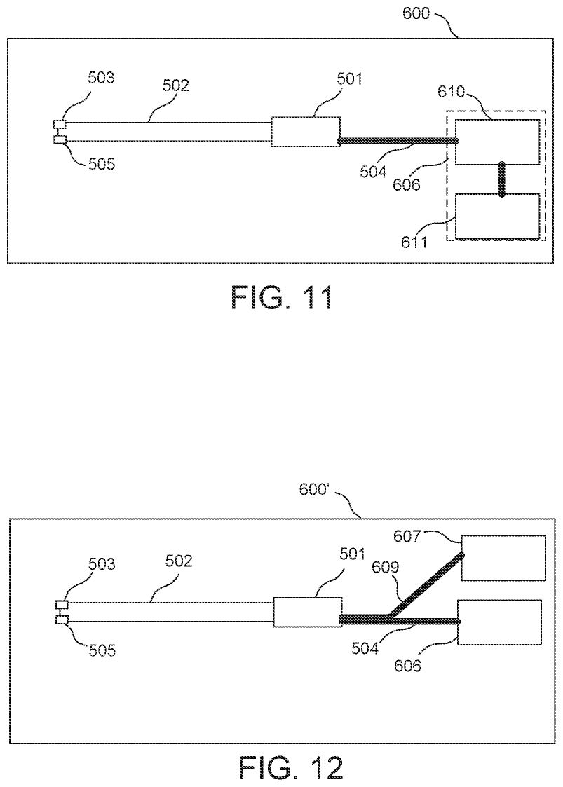

FIGS. 10A and 10B show schematically an embodiment of an endoscope 500 provided with an ultrasound transducer 505 according to the third aspect of the disclosure. FIG. 10A shows a view of the endoscope and FIG. 10B shows a close-up of a part of the ultrasound transducer 505. The endoscope 500 comprises a handle 501, an insertion tube 502 having a proximal end 510 and a distal end 511, a first flexible cable 504 and the ultrasound transducer 505 for receiving and transmitting ultrasound. The ultrasound transducer 505 comprises an ultrasound transducer array 520 attached to the distal end 511 of the insertion tube 502, the handle 501 being attached to the proximal end 510 of the insertion tube 502, the first flexible cable 504 extending from the handle 501 and being electrically connected to the ultrasound transducer array 520 and connectable to an ultrasound processing apparatus (shown in FIGS. 11 and 12). The first flexible cable 504 is configured to provide the ultrasound processing apparatus with electrical signals generated by the ultrasound transducer array 520 and to receive electrical signals generated by the ultrasound processing apparatus for the ultrasound transducer array.

The ultrasound transducer array 520 comprises a plurality of ultrasound transducer elements, an electrical circuit 530-533 for receiving and providing electrical signals to the plurality of ultrasound transducer elements, and a support structure 540 having an upper surface facing the plurality of ultrasound transducer elements and extending in a direction parallel to the first axis (described above). The support structure 540 may be the same as the support structure 101.

The electrical circuit 530-533 comprises a flexible base section 530, a plurality of first flexible electrical conductors 531-533, and a contact section 550. The individual first flexible electrical conductors are not shown. The flexible base section 530 comprises a plurality of first electrical contacts (not shown) each first electrical contact being connected to an ultrasound transducer element for providing and receiving electrical signals to/from the ultrasound transducer element and extending along a central axis (not shown) being parallel to the first axis, the flexible base section 530 being arranged on the upper surface of the support structure 540. Each of the plurality of first flexible electrical conductors 531-533 are electrically connected to a first electrical contact.

The contact section 550 comprises a plurality of second electrical contacts, each second electrical contact being electrically connected to a first electrical conductor 530-533 and further electrically connected or connectable to a second electrical conductor or a transducer port of an ultrasound processing apparatus. The flexible base section 530 and the plurality of first flexible electrical conductors 531-533 may be a single flexible printed circuit and the first flexible electrical conductors 531-533 may extend from the flexible base section 530 and into the handle 501. In this embodiment, the plurality of first flexible electrical conductors 531-533 for a first part of their length 531 extend inside the insertion tube 502, for a second part of their length 532 extend inside the handle 501, and for a third part of their length 533 extend inside the first flexible cable. The endoscope 500 further comprises an image sensor 503 shown in FIGS. 11 and 12. An example endoscope comprising an image sensor and ultrasound transducer, a handle, and a steering mechanism is describe in commonly owned U.S. patent application Ser. No. 18/210,882, filed Jun. 16, 2023, the disclosure of said application is incorporated by reference herein in its entirety.

FIGS. 11 and 12 show schematically a system 600 and a system 600′, both comprising the endoscope 500 including the image sensor 503 and the ultrasound transducer 505. Referring to FIG. 11, the system 600 comprises an ultrasound processing apparatus 606. The first flexible cable 504 extends from the handle 501 and is electrically connected to the ultrasound transducer 505 and to the ultrasound processing apparatus 606 and is configured to provide the ultrasound processing apparatus 606 with electrical signals generated by the ultrasound transducer 505 and receive electrical signals generated by the ultrasound processing apparatus 606 for the ultrasound transducer 505. The first flexible cable 504 is further configured to receive electrical signals from the image capturing device 503 and provide the received electrical signals to the ultrasound processing apparatus 606. The ultrasound processing apparatus 606 comprises a first processing unit, or ultrasound transducer controller, 610 configured to process electrical signals generate by the ultrasound transducer 505 and a second processing unit, or image sensor controller, 511 configured to process image signals recorded by the image capturing device 503.

Referring to FIG. 12, the system 600′ is similar to the system 600 except that the ultrasound processing apparatus 606 is different. The first flexible cable 504 extends from the handle 501 and is electrically connected to the ultrasound transducer 505 and to the ultrasound processing apparatus 606 and is configured to provide the ultrasound processing apparatus 606 with electrical signals generated by the ultrasound transducer 505 and receive electrical signals generated by the ultrasound processing apparatus 606 for the ultrasound transducer 505. The ultrasound processing apparatus 606 may be a conventional apparatus. A second flexible cable 609 is configured to receive electrical signals from the image capturing device 503 and provide the received electrical signals to an imaging unit, or video processing apparatus, 609. Example video processing apparatus are described in commonly owned U.S. Pat. Nos. 11,109,741 and 11,583,164, both incorporated by reference herein in their entirety.

Embodiments of the present disclosure, variations thereof, and examples thereof are set out in the following items:
1. A method of manufacturing a curvilinear ultrasound transducer for use with an endoscope, the method comprising: providing a piezoelectric block having an upper side and a lower side opposite to the upper side; providing a first acoustic matching layer; cutting the piezoelectric block at a plurality of positions forming a plurality of ultrasound transducer elements separated by a plurality of gaps; allowing the plurality of gaps to be filled with a first gas resulting in a plurality of gas-filled gaps; arranging the first acoustic matching layer above the piezoelectric block, optionally after the piezoelectric block has been cut; providing a support structure having a curvilinear upper surface; arranging the piezoelectric block with the first acoustic matching layer above the curvilinear upper surface of the support structure, optionally after the piezoelectric block has been cut; providing a pressure element; and moving the support structure and the pressure element together to shape the piezoelectric block and first acoustic matching layer curvilinear.
2. A method of manufacturing a curvilinear ultrasound transducer according to item 1, wherein the first gas is atmospheric air.
3. A method of manufacturing a curvilinear ultrasound transducer according to items 1 or 2, wherein the first acoustic matching layer is arranged above the plurality of ultrasound transducer elements after the piezoelectric block has been cut forming the plurality of ultrasound transducer elements.
4. A method of manufacturing a curvilinear ultrasound transducer according to item 3, wherein the method further comprising: providing a common ground electrode, and arranging the common ground electrode above the plurality of ultrasound transducer elements and subsequently arranging the first acoustic matching layer above the common ground electrode.
5. A method of manufacturing a curvilinear ultrasound transducer according to items 1 or 2, wherein the first acoustic matching layer is arranged above the piezoelectric block before the piezoelectric block has been cut.
6. A method of manufacturing a curvilinear ultrasound transducer according to item 5, wherein the method further comprising: providing a common ground electrode, and arranging the common ground electrode above the piezoelectric block and subsequently arranging the first acoustic matching layer above the common ground electrode.
7. A method of manufacturing a curvilinear ultrasound transducer according to any one of items 1 to 6, wherein the method further comprising: providing a flexible electrical circuit for electrically receiving and providing electrical signals to the plurality of ultrasound transducer elements wherein the flexible electrical circuit comprising a plurality of first electrical contacts each first electrical contact being connectable to an ultrasound transducer element for providing and receiving electrical signals to the ultrasound transducer element; and attaching the flexible electrical circuit to the lower side of the piezoelectric block before moving the support structure and pressure element together.
8. A method of manufacturing a curvilinear ultrasound transducer according to item 7, wherein the flexible electrical circuit is attached to the lower side of the piezoelectric block before the piezoelectric block has been cut, and wherein cutting of the piezoelectric block electrically insulates the plurality of first electrical contacts.
9. A curvilinear ultrasound transducer for use in connection with an endoscope procedure, wherein the curvilinear ultrasound transducer comprises an ultrasound transducer array comprising a plurality of ultrasound transducer elements made of a piezoelectric material, a support structure having a curvilinear upper surface, and one or more acoustic matching layers, the ultrasound transducer array being arranged above the curvilinear upper surface of the support structure, the one or more acoustic matching layers being arranged above the ultrasound transducer array and wherein a gas-filled gap is provided between each of the plurality of ultrasound transducer elements resulting in a plurality of gas-filled gaps.
10. A curvilinear ultrasound transducer according to item 9, wherein the ultrasound transducer further comprises an acoustic lens, the acoustic lens being formed as a single element and being arranged above the first acoustic matching layer.
11. A curvilinear ultrasound transducer according to items 9 or 10, wherein each ultrasound transducer element of the plurality of ultrasounds transducer elements is provided with an individual acoustic matching layer.
12. A curvilinear ultrasound transducer according to items 9 to 10, further comprising a common acoustic matching layer being arranged above the ultrasound transducer array.
13. A curvilinear ultrasound transducer according to any one of items 9 to 12, further comprising a flexible electrical circuit comprising a plurality of first electrical contacts each first electrical contact being connected to an ultrasound transducer element for providing and receiving electrical signals to the ultrasound transducer element, the flexible electrical circuit being arranged between the curvilinear upper surface of the support structure and the plurality of ultrasound transducer elements.
15. A curvilinear ultrasound transducer manufactured according to any one of items 1 to 8.
16. An ultrasound transducer comprising: a convex ultrasound transducer array comprising a plurality of ultrasound transducer elements; a support structure having a convex upper surface facing the plurality of ultrasound transducer elements and extending in a direction parallel to a first axis; and an electrical circuit configured to receive and provide electrical signals to the plurality of ultrasound transducer elements, the electrical circuit comprising: a base section that is flexible and comprises a plurality of first electrical contacts, each first electrical contact of the plurality of first electrical contacts connected to an ultrasound transducer element of the plurality of ultrasound transducer elements and extending along a central axis parallel to the first axis, the base section arranged on the convex upper surface of the support structure; a plurality of first electrical conductors, each first electrical conductor of the plurality of first electrical conductors electrically connected to a first electrical contact of the plurality of first electrical contacts; and a contact section comprising a plurality of second electrical contacts, each second electrical contact of the plurality of second electrical contacts electrically connected to a first electrical conductor of the plurality of first electrical conductors and further electrically connected or connectable to a second electrical conductor or a transducer port of an ultrasound processing apparatus, wherein a first group of the plurality of first electrical conductors are bent around the support structure at a first curve section and a second group of the plurality of first electrical conductors are bent around the support structure at a second curve section, the first curve section being separate from the second curve section.
17. The ultrasound transducer of item 16, wherein the base section and the plurality of first flexible conductors are comprised in a single flexible printed circuit.
18. The ultrasound transducer of item 16, wherein the support structure comprises a first side surface arranged in a plane perpendicular to the first axis, the first side surface connected to the convex upper surface, and wherein the first curve section is arranged along the connection between the convex upper surface and the first side surface.
19. The ultrasound transducer of item 18, wherein the second curve section is arranged along the connection between the convex upper surface and the first side surface and divided from the first curve section by a gap.
20. The ultrasound transducer of item 18, wherein the plurality of first conductors comprises a first group of electrical conductors and a second group of electrical conductors, the support structure comprises a second side surface opposite to the first side surface, the second side surface is arranged in a plane perpendicular to the first axis, the second side surface is connected to the convex upper surface, the second curve section is arranged along the connection between the convex upper surface and the second side surface, and wherein the first group of electrical conductors are bent around the first curve section and the second group of electrical conductors are bent around the second curve section.
21. An ultrasound transducer (505) for receiving and transmitting ultrasound comprising a convex ultrasound transducer array comprising a plurality of ultrasound transducer elements (180), an electrical circuit for receiving and providing electrical signals to the plurality of ultrasound transducer elements (180), and a support structure (101) having a convex upper surface (102) facing the plurality of ultrasound transducer elements (180) and extending in a direction parallel to a first axis (191), wherein the electrical circuit comprises: a flexible base section (103) comprising a plurality of first electrical contacts (110-122) each first electrical contact (110-122) being connected to an ultrasound transducer element for providing and receiving electrical signals to/from the ultrasound transducer element and extending along a central axis (193) being parallel to the first axis (191), the flexible base section (103) being arranged on the convex upper surface (102) of the support structure (101); a plurality of first flexible electrical conductors (150-162) each first flexible electrical conductor being electrically connected to a first electrical con-tact (110122); a contact section comprising a plurality of second electrical contacts each second electrical contact being electrically connected to a first electrical conductor (150-162) and further electrically connected or connectable to a second electrical conductor or a transducer port of an ultrasound processing apparatus, wherein the plurality of first flexible electrical conductors (150-162) are bent around the support structure (101) at a first curve section (140) and at a second curve section (141), the first curve section (140) being separate from the second curve section (141).
22. An ultrasound transducer according to item 21, wherein the support structure (101) has a first side surface (104) arranged in a plane being perpendicular to the first axis (191), the first side surface (104) being connected to the convex upper surface (102) and wherein the first curve section (140) is arranged along the connection between the convex upper surface (102) and the first side surface (104).
23. An ultrasound transducer according to item 22, wherein the second curve section (141) is arranged along the connection between the convex upper surface and the first side surface (104) and divided from the first curve section (140) by a gap (142).
24. An ultrasound transducer according to item 23, wherein the support structure comprises a concave lower surface (106) opposite to the convex upper surface (102), the first side surface (104) is connected to the concave lower surface (106), the gap (142) extends along the entire first side (104) of the support structure (101), and wherein the plurality of the first flexible electrical conductors (150-162) are further bent around the support structure at a third curve section (143) and at a fourth curve section (144), the third curve section (143) being separate from the fourth curve section (144) by the gap (142), and wherein the third curve section (143) and the fourth curve section (144) are arranged along the connection between the first side surface (104) and the concave lower surface (106).
25. An ultrasound transducer according to item 22, wherein the plurality of first flexible electrical conductors comprises a first group of flexible electrical conductors (150-156) and a second group of flexible electrical conductors (157-162), the support structure (101) has a second side surface (105) opposite to the first side surface (104), the second side surface (105) arranged in a plane being perpendicular to the first axis (191), the second side surface (105) being connected to the convex upper surface (102), the second curve section (141) is arranged along the connection between the convex upper surface (102) and the second side surface (105), and wherein the first group of flexible electrical conductors (150-156) are bent around the first curve section (140) and the second group of flexible electrical conductors (157-162) are bent around the second curve section (141).
26. An ultrasound transducer according to item 25, wherein the first group of flexible electrical conductors (150-156) are arranged in a first plane along a part of their length, the second group of flexible electrical conductors (157-162) are arranged in a second plane along a part of their length, where the first plane is arranged above the second plane.
27. An ultrasound transducer according to any one of item 21 to 26, wherein each of the plurality of second electrical contacts are connected to a second electrical conductor.
28. An ultrasound transducer according to item 27, wherein for each of the plurality of second electrical contacts the second electrical conductor is a coaxial cable permanently connected the second electrical contact.
29. An ultrasound transducer according to any one of item 21 to 26, wherein each of the plurality of second electrical contacts are configured to be detachable connectable to a second electrical conductor or a transducer port of an ultrasound processing apparatus.
30. An ultrasound transducer according to any one of items 21 to 29, wherein the flexible base section and the plurality of first flexible electrical conductors is a single flexible printed circuit.
31. A printed flexible electrical circuit for receiving and providing electrical signals to the plurality of ultrasound transducer elements, the printed flexible electrical circuit comprising: a flexible base section (103) comprising a plurality of first electrical contacts (110-122) each first electrical contact (110-122) being connectable to an ultrasound transducer element for providing and receiving electrical signals to the ultrasound transducer element and extending along a central axis (193) being parallel to a first axis (191); a plurality of first flexible electrical conductors (150-162) each first flexible electrical conductor being electrically connected to a first electrical contact (110-122); a contact section comprising a plurality of second electrical contacts each second electrical contact being electrically connected to a first electrical conductor (150-162) and further electrical connected or connectable to a second electrical conductor or a transducer port of an ultrasound processing apparatus, wherein the plurality of first flexible electrical conductors (150-162) is bendable around a support structure (101) at a first curve section (140) and at a second curve section (141), the first curve section (140) being separate from the second curve section (141).
32. An endoscope comprising a handle, an insertion tube, an image capturing device, a first flexible cable and an ultrasound transducer according to any one of items 9 to 13 or 21 to 31, the handle being attached to a proximal end of the insertion tube, the image capturing device and the ultrasound transducer being attached to a distal end of the insertion tube, and the first flexible cable is extending from the handle and being electrically connected to the plurality of ultrasound transducer elements via the electrical circuit and wherein the first flexible cable is connectable to an ultrasound processing apparatus and configured to provide the ultrasound processing apparatus with electrical signals generate by the plurality of ultrasound transducer elements and receive electrical signals generated by the ultrasound processing apparatus for the plurality of ultrasound transducer elements.
33. A system comprising an endoscope according to item 32 and an ultrasound processing apparatus, wherein the first flexible cable is connectable to the ultrasound processing apparatus.
34. A system according to item 33, wherein the first flexible cable is further configured to receive electrical signals from the image capturing device and provide the received electrical signals to the ultrasound processing apparatus, and wherein the ultrasound processing apparatus comprise one or more processing units configured to process both the signals received from the ultrasound transducer and the signals received from the image capturing device.
35. A system according to item 33, wherein the system further comprises an video processing apparatus and wherein the endoscope further comprises a second flexible cable configured to receive electrical signals from the image capturing device and provide the received electrical signals to the video processing apparatus.
36. An endoscope comprising an image capturing device and an ultrasound transducer according to items 9 to 30.
37. A printed flexible electrical circuit comprising: a flexible base section comprising a plurality of first electrical contacts, each first electrical contact being connectable to an ultrasound transducer element for providing and receiving electrical signals to/from the ultrasound transducer element and extending along a central axis parallel to a first axis; a plurality of first flexible conductors, each first flexible electrical conductor being electrically connected to a first electrical contact; and a contact section comprising a plurality of second electrical contacts, each second electrical contact being electrically connected to a first electrical conductor and further electrical connected or connectable to a second electrical conductor or a transducer port of an ultrasound processing apparatus, wherein the plurality of first flexible conductors is bendable around a support structure at a first curve section and at a second curve section, the first curve section being separate from the second curve section.
38. An endoscope comprising the printed flexible electrical circuit of item 37.
39. An endoscope comprising: an insertion tube having a proximal end and a distal end; a handle attached to the proximal end of the insertion tube; an ultrasound transducer attached to the distal end of the insertion tube and comprising an electrical circuit, a support structure, and an ultrasound transducer array; and a first flexible cable electrically connected to the ultrasound transducer array and connectable to an ultrasound processing apparatus, the first flexible cable being configured to provide the ultrasound processing apparatus with electrical signals generated by the ultrasound transducer array and to provide electrical signals generated by the ultrasound processing apparatus to the ultrasound transducer array, wherein the ultrasound transducer array comprises a plurality of ultrasound transducer elements, wherein the support structure comprises an upper surface facing the plurality of ultrasound transducer elements and extending in a direction parallel to a first axis, wherein the electrical circuit is configured to receive from and provide electrical signals to the plurality of ultrasound transducer elements and comprises: a flexible base section comprising a plurality of first electrical contacts, each first electrical contact being connected to an ultrasound transducer element of the plurality of ultrasound transducer elements and extending along a central axis that is parallel to the first axis, the flexible base section being arranged on the upper surface of the support structure, a plurality of first flexible electrical conductors, each first flexible electrical conductor being electrically connected to a first electrical contact, and a contact section comprising a plurality of second electrical contacts, each second electrical contact being electrically connected to a first electrical conductor and further electrically connected or connectable to a second electrical conductor or a transducer port of an ultrasound processing apparatus, wherein the flexible base section and the plurality of first flexible electrical conductors is a single flexible printed circuit, and wherein the first flexible electrical conductors extend from the flexible base section and into the handle.
40. The endoscope of item 39, wherein the plurality of first flexible electrical conductors for a first part of their length extend inside the insertion tube, for a second part of their length extend inside the handle, and for a third part of their length extend inside the first flexible cable.
41. An endoscope comprising a handle, an insertion tube having a proximal end and a distal end, a first flexible cable and an ultrasound transducer for receiving and transmitting ultrasound, the ultrasound transducer comprising an ultrasound transducer array attached to the distal end of the insertion tube, the handle being attached to the proximal end of the insertion tube, the first flexible cable is extending from the handle and being electrically connected to the ultrasound transducer array and is connectable to an ultrasound processing apparatus and configured to provide the ultrasound processing apparatus with electrical signals generated by the ultrasound transducer array and receive electrical signals generated by the ultrasound processing apparatus for the ultrasound transducer array, and wherein the ultrasound transducer array comprises a plurality of ultrasound transducer elements (180), and wherein the ultrasound transducer comprises an electrical circuit for receiving and providing electrical signals to the plurality of ultrasound transducer elements (180), and a support structure (101) having an upper surface (102) facing the plurality of ultrasound transducer elements (180) and extending in a direction parallel to a first axis (191), wherein the electrical circuit comprises: a flexible base section (103) comprising a plurality of first electrical contacts (110-122) each first electrical contact (110-122) being connected to an ultrasound transducer element for providing and receiving electrical signals to/from the ultrasound transducer element and extending along a central axis (193) being parallel to the first axis (191), the flexible base section (103) being arranged on the upper surface (102) of the support structure (101); a plurality of first flexible electrical conductors (150-162) each first flexible electrical conductor being electrically connected to a first electrical contact (110-122); and a contact section comprising a plurality of second electrical contacts each second electrical contact being electrically connected to a first electrical conductor (150-162) and further electrically connected or connectable to a second electrical conductor or a transducer port of an ultrasound processing apparatus, wherein the flexible base section and the plurality of first flexible electrical conductors is a single flexible printed circuit and wherein the first flexible electrical conductors (150-162) extend from the flexible base section (103) and into the handle.
42. An endoscope according to item 41, wherein the plurality of first flexible electrical conductors (150-162) for a first part of their length extends inside the insertion tube, for a second part of their length extends inside the handle, and for a third part of their length extend inside the first flexible cable.
43. An endoscope according to item 42, wherein the first flexible cable comprises an ultrasound connector insertable into a transducer port of an ultrasound processing apparatus for providing electrical signals generated by the ultrasound transducer array to the ultrasound processing apparatus and receiving electrical signals generated by the ultrasound processing apparatus, wherein the plurality of first flexible electrical conductors (150-162) extend throughout the entire length of the first flexible cable and the ultrasound connector is the contact section.
44. An endoscope according to item 43, wherein the endoscope further comprises an image capturing device and wherein the first flexible cable is further configured to receive electrical signals from the image capturing device.
45. An endoscope according to any one of items 41 to 44, wherein the plurality of first flexible electrical conductors (150-162) are folded at least once to extend their length.
46. An endoscope according to any one of items 41 to 45, wherein the upper surface (102) is a convex upper surface, the plurality of first flexible electrical conductors (150-162) are bent around the support structure (101) at a first curve section (140) and at a second curve section (141), the first curve section (140) being separate from the second curve section (141).
47. An endoscope according to item 6, wherein the support structure (101) has a first side surface (104) arranged in a plane being perpendicular to the first axis (191), the first side surface (104) being connected to the convex upper surface (102) and wherein the first curve section (140) is arranged along the connection between the convex upper surface (102) and the first side surface (104).
48. An endoscope according to item 47, wherein the second curve section (141) is arranged along the connection between the convex upper surface and the first side surface (104) and divided from the first curve section (140) by a gap (142).
49. An endoscope according to item 48, wherein the support structure comprises a concave lower surface (106) opposite to the convex upper surface (102), the first side surface (104) is connected to the concave lower surface (106), the gap (142) extends along the entire first side (104) of the support structure (101), and wherein the plurality of the first flexible electrical conductors (150-162) are further bent around the support structure at a third curve section (143) and at a fourth curve section (144), the third curve section (143) being separate from the fourth curve section (144) by the gap (142), and wherein the third curve section (143) and the fourth curve section (144) are arranged along the connection between the first side surface (104) and the concave lower surface (106).
50. A printed flexible electrical circuit for receiving and providing electrical signals to an ultrasound transducer array comprising a plurality of ultrasound transducer elements, the printed flexible electrical circuit comprising: a flexible base section (103) comprising a plurality of first electrical contacts (110-122) each first electrical contact (110-122) being connectable to an ultrasound transducer element for providing and receiving electrical signals to the ultrasound transducer element and extending along a central axis (193) being parallel to a first axis (191); and a plurality of first flexible electrical conductors (150-162) each first flexible electrical conductor being electrically connected to a first electrical contact (110-122), wherein the plurality of first flexible electrical conductors (150-162) are connectable to an ultrasound connector whereby the first flexible electrical conductors may be responsible for the entire electrical coupling between the ultrasound connector and the ultrasound transducer array.
51. A printed flexible electrical circuit wherein the plurality of first flexible electrical conductors (150-162) have a length of at least 40 cm.
52. A system comprising an endoscope according to any one of items 38 to 49 and an ultrasound processing apparatus, wherein the endoscope comprises an image capturing device and the first flexible cable is connectable to the ultrasound processing apparatus.
53. A system according to item 52, wherein the first flexible cable is further configured to receive electrical signals from the image capturing device and provide the received electrical signals to the ultrasound processing apparatus, and wherein the ultrasound processing apparatus comprise one or more processing units configured to process both the signals received from the ultrasound transducer and the signals received from the image capturing device.
54. A system according to item 53, wherein the system further comprises a video processing apparatus and wherein the endoscope further comprises a second flexible cable configured to receive electrical signals from the image capturing device and provide the received electrical signals to the video processing apparatus.

Although some embodiments have been described and shown in detail, the invention is not restricted to them, but may also be embodied in other ways within the scope of the subject matter defined in the following items. In particular, it is to be understood that other embodiments may be utilized and structural and functional modifications may be made without departing from the scope of the present invention.

In device claims enumerating several means, several of these means can be embodied by one and the same item of hardware. The mere fact that certain measures are recited in mutually different dependent claims or described in different embodiments does not indicate that a combination of these measures cannot be used to advantage.

The terms “comprises/comprising,” “includes/including,” “having/have,” and derivatives thereof are inclusive transition terms that describe the presence of stated features, integers, steps or components but do not preclude the presence or addition of one or more other features, integers, steps, components or groups thereof.

1. A curvilinear ultrasound transducer for an endoscope, the curvilinear ultrasound transducer comprising:
a plurality of ultrasound transducer elements made of a piezoelectric material;
a support structure having a curvilinear upper surface; and
an acoustic matching layer, the plurality of ultrasound transducer elements arranged between the curvilinear upper surface of the support structure and the acoustic matching layer,
wherein the plurality of ultrasound transducer elements comprises ultrasound transducer elements separated by gas-filled gaps between the ultrasound transducer elements.

2. The curvilinear ultrasound transducer of claim 1, wherein the ultrasound transducer further comprises an acoustic lens facing the acoustic matching layer, the acoustic lens being formed as a single element.

3. The curvilinear ultrasound transducer of claim 1, the acoustic matching layer is comprised of acoustic matching layer portions, and wherein each of the ultrasound transducer elements is provided with an acoustic matching layer portion from the acoustic matching layer portions.

4. The curvilinear ultrasound transducer of claim 3, further comprising a common acoustic matching layer facing the acoustic matching layer portions.

5. The curvilinear ultrasound transducer of claim 4, further comprising a flexible electrical circuit comprising first electrical contacts, each of the first electrical contacts connected to an ultrasound transducer element of the plurality of ultrasound transducer elements, the flexible electrical circuit arranged between the curvilinear upper surface of the support structure and the plurality of ultrasound transducer elements.

6. The curvilinear ultrasound transducer of claim 5, further comprising a common ground electrode between the plurality of ultrasound transducer elements and the acoustic matching layer.

7. The curvilinear ultrasound transducer of claim 1, further comprising a common acoustic matching layer facing the ultrasound transducer elements.

8. The curvilinear ultrasound transducer of claim 1, further comprising a flexible electrical circuit comprising first electrical contacts, each of the first electrical contacts connected to an ultrasound transducer element of the plurality of ultrasound transducer elements, the flexible electrical circuit arranged between the curvilinear upper surface of the support structure and the plurality of ultrasound transducer elements.

9. The curvilinear ultrasound transducer of claim 8, further comprising a common ground electrode between the plurality of ultrasound transducer elements and the acoustic matching layer.

10. The curvilinear ultrasound transducer of claim 1, the curvilinear ultrasound transducer further comprising an acoustic lens facing the acoustic matching layer; a flexible electrical circuit comprising first electrical contacts, each of the first electrical contacts connected to an ultrasound transducer element of the plurality of ultrasound transducer elements, the flexible electrical circuit arranged between the curvilinear upper surface of the support structure and the plurality of ultrasound transducer elements; and a common ground electrode between the plurality of ultrasound transducer elements and the acoustic matching layer.

11. The curvilinear ultrasound transducer of claim 10, wherein the flexible electrical circuit further comprises a plurality of first flexible electrical conductors, each first flexible electrical conductor of the plurality of first flexible electrical conductors being electrically connected to a first electrical contact of the first electrical contacts, wherein the first flexible electrical conductors are bent around the support structure at a first curve section and at a second curve section, the first curve section being separate from the second curve section.

12. The curvilinear ultrasound transducer of claim 11, wherein a first group of the first flexible electrical conductors are bent around the support structure at the first curve section and a second group of the first flexible electrical conductors are bent around the support structure at the second curve section, the first curve section being separate from the second curve section.

13. The curvilinear ultrasound transducer of claim 12, wherein the first curve section extends along a first edge of the curvilinear upper surface and the second curve section extends along a second edge of the curvilinear upper surface, the first edge being opposite the second edge.

14. The curvilinear ultrasound transducer of claim 12, wherein the first curve section and the second curve section extend along a first edge of the curvilinear upper surface.

15. An endoscope comprising: an image sensor; and a curvilinear ultrasound transducer according to claim 1.

16. A method of manufacturing the curvilinear ultrasound transducer of claim 1, the method comprising:
providing a piezoelectric block having an upper side and a lower side opposite to the upper side;
cutting the piezoelectric block at a plurality of positions forming the plurality of ultrasound transducer elements, the plurality of ultrasound transducer elements separated by a plurality of gaps;
allowing the plurality of gaps to be filled with a first gas resulting in a plurality of gas-filled gaps;
arranging the acoustic matching layer facing the upper side of the piezoelectric block;
arranging the piezoelectric block and the first acoustic matching layer between the curvilinear upper surface of the support structure and a pressure element; and
pressing the piezoelectric block and the first acoustic matching layer with the support structure and the pressure element to curvilinearly shape the piezoelectric block and first acoustic matching layer.

17. The method of claim 16, wherein the arranging of the first acoustic matching layer facing the upper side of the piezoelectric block is performed after cutting the piezoelectric block.

18. The method of claim 16, wherein the first gas is atmospheric air.

19. The method of claim 16, wherein cutting the piezoelectric block forms the plurality of ultrasound transducer elements, and wherein the acoustic matching layer is arranged facing the plurality of ultrasound transducer elements.

20. The method of claim 19, further comprising arranging a common ground electrode facing the plurality of ultrasound transducer elements, and after arranging the common ground electrode, arranging the first acoustic matching layer facing the common ground electrode.

21. The method of claim 16 wherein the arranging of the first acoustic matching layer facing the upper side of the piezoelectric block is performed before cutting the piezoelectric block.

22. The method of claim 21, the method further comprising arranging a common ground electrode facing the piezoelectric block and, after the common ground electrode, arranging the first acoustic matching layer facing the common ground electrode.

23. The method of claim 16, the method further comprising:
providing a flexible electrical circuit comprising a plurality of first electrical contacts, each first electrical contact being connectable to an ultrasound transducer element of the plurality of ultrasound transducer elements; and attaching the flexible electrical circuit to the lower side of the piezoelectric block before pressing the piezoelectric block and the first acoustic matching layer with the support structure and the pressure element.

24. The method of claim 23, wherein the flexible electrical circuit is attached to the lower side of the piezoelectric block before the piezoelectric block has been cut, and wherein cutting of the piezoelectric block electrically insulates the plurality of first electrical contacts.
